(19) United States
(12) Patent Application Publication
(10) Pub. No.: US 2025/0073168 A1
(43) Pub. Date: Mar. 6, 2025

(54) GELLAN GUM COMPOSITIONS AND METHOD FOR THEIR PREPARATION

(71) Applicant: Auregen Biotherapeutics SA, Geneva (CH)

(72) Inventors: Matti Jaakko Johannes Kesti, Zurich (CH); Gary Yewey, Geneva (CH)

(21) Appl. No.: 18/570,958

(22) PCT Filed: Jun. 14, 2022
(86) PCT No.: PCT/EP2022/066099

§ 371 Date: Dec. 15, 2023

(30) Foreign Application Priority Data
Jun. 16, 2021 (EP) 21179842.6

(51) Int. Cl. A61K 9/16; A61K 9/19

(52) U.S. Cl. CPC A61K 9/1652; A61K 9/1617; A61K 9/1694; A61K 9/19

(57) ABSTRACT

The present invention generally relates to the field of biopolymers. In one aspect. the invention is directed to a method of preparing lyophilized and reconstituted gellan gum compositions. In further aspects. the invention relates to lyophilized compositions comprising gellan gum, reconstituted gellan gum compositions. dosage-unit formulations comprising lyophilized gellan gum, and their use in medicine or other sterile applications.

### DESCRIPTION

### Field of the Invention

The present invention generally relates to the field of biopolymers. In one aspect, the invention is directed to a method of preparing lyophilized and reconstituted gellan gum compositions. In further aspects, the invention relates to lyophilized compositions comprising gellan gum, reconstituted gellan gum compositions, dosage-unit formulations comprising lyophilized gellan gum, and their use in medicine or other sterile applications.

### Background of the Invention

Gellan gum is a polysaccharide product obtained via fermentation of a bacterial strain of the genus Sphingomonas. It is a linear anionic polysaccharide composed of tetrasaccharide repeating units. Each tetrasaccharide includes one β-D-glucuronic acid monomer, one α-L-rhamnose monomer and two β-D-glucose monomers. The chemical formula is shown in FIG. 1. In its native form, one of the glucose monomers contains an O-linked acetyl and an O-linked glyceryl moiety, and is commonly referred to as high acyl gellan gum (FIG. 1(a)). Exposure to alkali at high temperature leads to removal of the acyl groups, yielding deacylated gellan gum, also commonly referred to as low acyl gellan gum (FIG. 1(b)). Gellan gum has been first described in the 1980s and is widely used as a food additive (E 418). In the last decade, additional uses in the pharmaceutical field have been proposed, for example in ophthalmic formulations, wound healing, in situ gelling systems, controlled release formulations and solid dosage forms. In addition, gellan gum is used in tissue engineering, for instance in cartilage repair. It has been described as a stabilizer and thickener in the process of 3D printing of biopolymer constructs containing living cells, see e.g. WO 2016/092106 A1 and EP 20 180 620.5, the contents of which are incorporated by reference herein. Gellan gum is not classified as dangerous (regulation EC 1272/2008 GHS).

Gellan gum powder is commercially available. However, despite its use in diverse applications in the pharmaceutical field indicated above, it is not commercially available in sterile quality. Sterilization thus has to be performed at the point of use. However, due to its viscosity, sterilization of products containing gellan gum is complicated. For a widespread use in emerging technologies such as bioprinting, it is crucial to dispose of gellan gum that meets current Good Manufacturing Practice (cGMP) regulations, in particular cGMP regulations for drug products to ensure the required product quality in a consistent and reproducible manner.

### BRIEF SUMMARY OF THE INVENTION

The present inventors have surprisingly found that with the use of a specific preparation method including a lyophilization step, it is possible to obtain gellan gum compositions that fulfill cGMP regulations, particularly cGMP regulations for drug products and thus can be used in medical and sterile applications.

Accordingly, the present invention is directed to a method of preparing a lyophilized gellan gum composition, comprising the steps of:
(i) Adding gellan gum powder to a pharmaceutically acceptable preparatory solvent;
(ii) Mixing until substantially complete dissolution;
(iii) Optionally adding further preparatory solvent;
(iv) Lyophilizing the obtained solution, thereby providing the lyophilized gellan gum composition;
   wherein the concentration of gellan gum after step (iii) is up to about 3 mg/ml.

The invention is also directed to a method of preparing a reconstituted gellan gum composition, comprising the steps of:
(i) Adding gellan gum powder to a pharmaceutically acceptable preparatory solvent;
(ii) Mixing until substantially complete dissolution;
(iii) Optionally adding further preparatory solvent;
(iv) Lyophilizing the obtained solution;
(v) Adding a sufficient amount of a pharmaceutically acceptable reconstitution solvent, thereby providing the reconstituted gellan gum composition;
   wherein the concentration of gellan gum after step (iii) is up to about 3 mg/ml.

For instance, the concentration of gellan gum after step (iii) may be from about 1 mg/ml to about 3 mg/ml. In some embodiments, the concentration of gellan gum after step (iii) is from about 1.0 mg/ml to about 3.0 mg/ml, particularly from about 1.5 mg/ml to about 2.5 mg/ml, more particularly about 2.0 mg/ml.

Dissolution of gellan gum powder can be achieved in a broad temperature range. Advantageously, the mixing step (ii) may be carried out at a temperature of max. 95° C. In particular embodiments, the mixing step (ii) is carried out between about 70° C.-95° C. or between about 25° C.-40° C.

According to the invention, it is in particular envisaged that the method further comprises at least one filtration step. This filtration step is suitably performed with the solution obtained in step (iii). Thus, in some embodiments, the solution obtained in step (iii) is filtered over a bioburden reducing filter and/or filter-sterilized, before being subjected to lyophilization. Particularly, the solution may first be subjected to bioburden reducing filtration and then subjected to sterile filtration, thereby providing a sterile solution, before being subjected to lyophilization in step (iv).

In certain embodiments, the gellan gum used in the method of the invention is low acyl gellan gum.

The pharmaceutically acceptable preparatory solvent may particularly be water or an aqueous solution of a calcium ion sequestrant, more particularly an alkali metal citrate, alkali metal phosphate or alkali metal malate solution, more particularly an aqueous sodium citrate solution, more particularly an aqueous solution of tri-sodium citrate dihydrate.

Where the preparatory solvent contains a calcium ion sequestrant, the concentration of the calcium ion sequestrant in the composition after step (iii) may in certain embodiments be from about 0.1 mM per gram gellan gum powder to about 0.2 mM per gram gellan gum powder, particularly about 0.15 mM per gram gellan gum powder.

In particular embodiments of the method according to the invention, the pharmaceutically acceptable preparatory solvent is an aqueous solution of sodium citrate, and the concentration of sodium citrate in the composition after step (iii) is from about 0.1 mM per gram gellan gum powder to about 0.2 mM per gram gellan gum powder, particularly about 0.15 mM per gram gellan gum powder.

In certain embodiments, the method of preparing a lyophilized composition according to the invention comprises the steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iv) Lyophilizing the obtained solution;
   wherein the concentration of gellan gum after step (iii) is from about 1 mg/ml to about 3 mg/ml.

The present invention is further directed to a lyophilized composition comprising gellan gum and a calcium ion sequestrant, wherein the weight ratio of gellan gum to calcium ion sequestrant is from about 15:1 to about 30:1, particularly from about 20:1 to about 27:1, more particularly from about 21.5:1 to about 25:1. In preferred embodiments, the lyophilized composition is sterile.

The gellan gum used in the lyophilized composition may be low or high acyl gellan gum. The calcium ion sequestrant may be selected from a variety of suitable compounds. In some embodiments, the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate.

In some embodiments, the moisture content of the composition is about 5% by weight or less, particularly about 4% by weight or less.

The invention is further directed to a dosage-unit formulation comprising a lyophilized composition according to any one of the preceding claims in a container with a volume sufficient to allow reconstitution of gellan gum with a pharmaceutically acceptable reconstitution solvent.

Still further, the invention is directed to a sterile composition reconstituted from a sterile lyophilized composition, comprising gellan gum and a calcium ion sequestrant, wherein the weight ratio of gellan gum to calcium ion sequestrant is from about 15:1 to about 30:1; and a pharmaceutically acceptable reconstitution solvent, wherein the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate.

Still further, the present invention is directed to medical uses of the compositions described above. Accordingly, the invention provides a lyophilized composition comprising gellan gum and a calcium ion sequestrant, wherein the weight ratio of gellan gum to calcium ion sequestrant is from about 15:1 to about 30:1, particularly from about 20:1 to about 27:1, more particularly from about 21.5:1 to about 25:1 for use in medicine, particularly human medicine. Also provided according to the invention is a sterile composition reconstituted from a sterile lyophilized composition, comprising gellan gum and a calcium ion sequestrant, wherein the weight ratio of gellan gum to calcium ion sequestrant is from about 15:1 to about 30:1; and a pharmaceutically acceptable reconstitution solvent, for use in medicine, particularly human medicine.

Finally, the invention is directed to the use of a lyophilized composition as described above, after reconstitution of the lyophilized composition comprising gellan gum and a calcium ion sequestrant such as sodium citrate, in sterile applications.

### DETAILED DESCRIPTION OF THE INVENTION

The present invention is, in a first aspect, concerned with providing a method of preparing a lyophilized gellan gum composition. The method comprises at least the steps described in the following.

In a first step (step (i)), gellan gum powder is added to a pharmaceutically acceptable preparatory solvent. In the context of the present invention, both high-acyl and low acyl gellan gum or any mixture thereof can be used. In preferred embodiments, low acyl gellan gum is used. The pharmaceutically acceptable preparatory solvent may be chosen according to the needs of the intended application. As already mentioned, the pharmaceutically acceptable preparatory solvent may particularly be water or an aqueous solution of a calcium ion sequestrant.

A “calcium ion sequestrant” in the context of the present invention is any substance used to bring about sequestration of calcium ions, typically by chelation. By the use of sequestrants, the amount of divalent ions in the solvent used can be controlled. Divalent calcium cations (Ca²⁺) inter alia can form crosslinks with gellan gum and thus a high concentration of such cations is undesired in the stage of preparation of the lyophilized composition. Various citrates, phosphates and malates possess sequestering properties for calcium ions and can be used as a according to the invention. In particular, the pharmaceutically acceptable calcium ion sequestrant is selected from aqueous solutions, i.e. solutions wherein the solvent is water, of an alkali metal citrate, an alkali metal phosphate or an alkali metal malate. Suitable alkali metals include potassium and sodium. In specific embodiments, the pharmaceutically acceptable calcium ion sequestrant is selected from aqueous solutions of a sodium citrate, a potassium citrate, a sodium phosphate, a potassium phosphate, a sodium malate, a potassium malate and pharmaceutically acceptable mixtures thereof. An exemplary calcium ion sequestrant according to the invention is an aqueous sodium citrate solution, more particularly an aqueous solution of tri-sodium citrate dihydrate.

In the second step of the method (step (ii)), the mixture obtained in step (i) is mixed until the gellan gum powder is substantially completely dissolved. In the context of gellan gum, “dissolved” or “dissolution” are also referred to as “hydrated” or “hydration”. “Substantially completely dissolved” as used herein means that less than about 0.1%, particularly less than about 0.01% of gellan gum based on the total amount of gellan gum added remains undissolved. In preferred embodiments, the gellan gum powder is completely dissolved, which can be verified, e.g., by visual inspection of the mixture (no remaining visible particles). Mixing can be performed by means well known to the skilled person and acceptable under cGMP regulations, for example by agitation via shakers or vortex or stirring via magnetic stirrers. “Obtained in” as used herein with reference to a process step means the product as present upon completion of the respective process step.

Depending on the composition of the preparatory solvent, it may be suitable or even required to use increased temperatures for complete dissolution of gellan gum powder. In particular, the mixing step (ii) may be carried out at a maximum of 95° C. In some embodiments, the mixing step (ii) is carried out between about 70° C. and about 95° C., for example at about 80° C. Temperatures in this more elevated range are particularly used where the preparatory solvent does not contain a calcium ion sequestrant. In other embodiments, the mixing step (ii) is carried out between about 25° C. and about 40° C. Temperatures in this range are particularly used where the preparatory solvent contains a calcium ion sequestrant.

Mixing at elevated temperatures, e.g. between about 70° C. and about 95° C., may be carried out for e.g. between 30 min and 60 min.

The inventors have surprisingly found that by adding a calcium ion sequestrant, e.g. sodium phosphate or sodium citrate, particularly sodium citrate, in a suitable, particularly a comparatively high amount (by using aqueous solutions containing, e.g., from about 1% by weight (also referred to herein as wt %) to about 6 wt %, particularly 3.5 wt % to 4.5 wt % calcium ion sequestrant, such as sodium citrate or sodium phosphate, particularly sodium citrate, relative to the total weight of gellan gum and calcium ion sequestrant), less or no external heating of the solvent is necessary to completely dissolve the gellan gum powder. Moreover, this reduces temperature strain on the containers used and issues with gelation in cooling and damage to the containers used (e.g. breakage of glass vials) can be reduced. On the other hand the suitable amounts of calcium ion sequestrant, particularly aqueous solutions containing, e.g., from about 1 wt % to about 6 wt %, particularly 3.5 wt % to 4.5 wt % calcium ion sequestrant, such as sodium citrate or sodium phosphate, particularly sodium citrate, relative to the total weight of gellan gum and calcium ion sequestrant, do not interfere with viscosity of the gellan gum required for downstream applications. It was further surprisingly found that despite the known pH lowering effect of calcium ion sequestrants on biopolymers, there is no interference with cell viability in downstream applications.

Exemplary temperatures within the range of 25° C.-40° C. are 30° C., 32° C., 34° C., 36° C., 38° C. External heating may be provided by means well known in the art, such as electrical heating blankets or jackets. This allows to reduce the equipment made from stainless steel and facilitates manufacturing. Accordingly, in certain preferred embodiments, a calcium ion sequestrant is used and the temperature applied for complete dissolution of gellan gum is between 25° C. and 40° C. In exemplary embodiments, an aqueous sodium citrate solution, particularly an aqueous solution of tri-sodium citrate dihydrate is used at a temperature of between 35° C. and 40° C., such as 36° C. or 38° C. In certain preferred embodiments, an aqueous solution of from 3.5 wt % to 4.5 wt % of sodium citrate, particularly an aqueous solution of tri-sodium citrate dihydrate, relative to the total weight of gellan gum and sodium citrate, particularly tri-sodium citrate dihydrate, is used at a temperature of between 35° C. and 40° C., such as 36° C. or 38° C.

Where heating is used, the heated solution is particularly cooled down, e.g to a temperature within the range of 25° C.-40° C. or to room temperature before proceeding further. For example, the hot solution may be cooled overnight at room temperature under continuous mixing.

After the gellan gum is dissolved in the preparatory solvent, further preparatory solvent may be added in step (iii). This may be required, e.g., to adjust the gellan gum concentration to a predetermined value, particularly if the concentration after step (ii) is above about 3 mg/ml. According to the invention, the gellan gum concentration after step (iii) is up to about 3 mg/ml. Typically, the further preparatory solvent is the same preparatory solvent as that used in step (i). Where all parameters of the solution obtained in step (ii) already meet predetermined requirements (e.g., the concentration of gellan gum is up to about 3 mg/ml), no further preparatory solvent will be added and the method continues with step (iv). In other words, step (iii) may serve to adjust the concentrations of gellan gum and/or calcium ion sequestrant as needed.

By the optional addition of further preparatory solvent, also the concentration of the calcium ion sequestrant, if present in the preparatory solvent, will be modified. In particular, the concentration of the calcium ion sequestrant in the composition after step (iii) is from about 0.1 mM per gram gellan gum powder to about 0.2 mM per gram gellan gum powder, particularly about 0.15 mM per gram gellan gum powder. According to some embodiments, the pharmaceutically acceptable preparatory solvent is an aqueous solution of sodium citrate, and the concentration of sodium citrate in the composition after step (iii) is from about 0.1 mM per gram gellan gum powder to about 0.2 mM per gram gellan gum powder, particularly about 0.15 mM per gram gellan gum powder.

In a further mandatory step (step (iv)), the solution obtained in step (iii) is subjected to lyophilization, thereby providing the lyophilized gellan gum composition. Lyophilization or freeze-drying is a well-known process in which water is removed from a given composition after it is frozen and placed under vacuum. Lyophilization can be subdivided into three phases: in the first phase, the solution obtained in step (iii) is subjected to freezing, followed by sublimation (primary drying) and desorption (secondary drying). According to the invention, particularly up to about 95% by weight or more, more particularly about 96 wt % or more, of the water is removed, i.e. the moisture content of the lyophilized composition after step (iv) is particularly about 5 wt % or less, more particularly about 4 wt % or less.

Freezing temperatures in accordance with the invention may for example range from about −10° C. to about −50° C., at atmospheric pressure. Primary drying may be performed in a temperature range of, e.g., about 0° C. to about −15° C. and reduced pressure, e.g. 1 mbar or less, such as 200 μbar or 100 μbar. Secondary drying may be performed in a temperature range of, e.g., about 0° C. to about +50° C. and reduced pressure, e.g. 1 mbar or less, such as 200 μbar or 100 μbar.

In a further aspect, the present invention relates to a method of preparing a reconstituted gellan gum composition, which comprises the steps described above with respect to the method of preparing the lyophilized gellan gum composition (i.e. step (i), step (ii), step (iii) and step (iv)) and additionally a step of adding a sufficient amount of a pharmaceutically acceptable reconstitution solvent, thereby providing the reconstituted gellan gum composition (step (v)).

The pharmaceutically acceptable reconstitution solvent may be selected from a wide variety of liquid compositions. However, it has to be taken into account that by lyophilization, only water has been removed and compounds previously dissolved in the preparatory solvent of step (i) will be redissolved in the reconstitution solvent. Further, the use intended for the reconstituted gellan gum composition will influence the choice of the reconstitution solvent. In some embodiments, the reconstitution solvent is water, particularly water for injection (WFI). In other embodiments, the reconstitution solvent is glucose solution. For instance, the glucose solution may be aqueous glucose solution with a glucose concentration of e.g. about 300 mM, which may be buffered, e.g. citrate-buffered. Solutions containing high amounts of cations that would interfere with the use of gellan gum in certain downstream applications, such as additive manufacturing, are excluded from being used as reconstituting solvent.

The amount of the reconstitution solvent used will be determined by the intended further use of the gellan gum composition. Where the gellan gum is, for instance, to be used in downstream applications such as additive manufacturing, particularly 3D printing, it is preferred that the concentration of gellan gum in the reconstituted composition is relatively high. According to some embodiments, the concentration of gellan gum in reconstitution solvent is between about 20 mg/ml and about 50 mg/ml. Specific exemplary concentrations of gellan gum in reconstitution solvent are 20 mg/ml, 25 mg/ml, 30 mg/ml, 35 mg/ml.

In preferred embodiments of the method of preparing a lyophilized gellan gum composition, the method further comprises at least one filtration step. Likewise, in preferred embodiments of the method of preparing a reconstituted gellan gum composition, the method further comprises at least one filtration step. Filtration can help in reducing the bioburden and removing undesired particles that have been introduced somewhere in the respective method. It can even constitute a possibility to provide sterile cGMP gellan gum. Filtration can in principle be performed in cartridges or single use filters using a variety of filter materials, such as activated carbon, fibres or membranes. In preferred embodiments of the present invention, membranes are used as filter materials. Suitable membrane filter materials according to the invention include polyethersulfone (PES) and polyvinylidene difluoride (PVDF).

In some embodiments of the invention, the composition obtained in step (iii) of the methods described above is subjected to sterile filtration. Suitable filters for producing sterile filtrate have a pore size of 0.22 μm or less, particularly 0.2 μm or less and are capable of passing a bacterial challenge to retain a minimum of 107 cfu/cm² of Brevundimonas dimunita. Some samples may benefit from even lower pore sizes, such as 0.1 μm pore size filtration. In some embodiments, a PVDF membrane with a pore size of 0.22 μm or less, e.g. 0.22 μm or 0.2 μm, is used for sterile filtration.

Alternatively or in addition, the composition obtained in step (iii) of the methods described above is, in some embodiments of the invention, subjected to bioburden reducing filtration. Suitable filters for bioburden reduction filtration according to the invention have a pore size of 0.4 μm or less, particularly 0.22 μm or less. For example, a PES membrane filter with a pore size of 0.4 μm or less, e.g. 0.22 μm or 0.2 μm, may be used for bioburden filtration. Bioburden reduction filtration as used herein preferably reduces the number of colony-forming units (CFU) to 10 per 100 mL.

In certain preferred embodiments of the invention, the solution obtained in step (iii) of the methods described above is subjected to both bioburden reducing filtration (also termed pre-filtration) and sterile filtration (also termed filter-sterilization). For example, if no more than 10 CFU/100 ml will be acceptable, depending on the volume to be filtered in relation to the diameter of the filter, and this requirement is not met, it may be necessary to use a pre-filtration through a bacteria-retaining filter to obtain a sufficiently low bioburden for subsequent sterile filtration. In certain preferred embodiments, the solution obtained in step (iii) is subjected to two filtration steps. It is accordingly further preferred that the solution obtained in step (iii) is first subjected to bioburden reducing filtration and then subjected to sterile filtration, thereby providing a sterile solution.

As used herein, the term “sterile” means free from viable microorganisms. For example, a composition may be defined as sterile if it has a sterility assurance level (SAL) equal to or less than 10⁻⁶. The SAL for a given sterilisation process is expressed as the probability of micro-organisms surviving in a product item after exposure to the process. An SAL of 10⁻⁶, for example, denotes a probability of not more than one non-sterile item in 1×10⁶ sterilised items of the final product. The inventors have found that filter-sterilization is advantageous over other sterilization methods such as γ-irradiation, because there is no risk to change the product composition by creating unwanted impurities, which may interfere with cGMP regulations, particularly cGMP regulations for drug products.

In particular in cases where the solution obtained in step (iii) is subjected to bioburden reducing filtration or sterile filtration, the lyophilization step (iv) is preferably performed under aseptic conditions. “Aseptic conditions” are those designed to prevent microbiological contamination. For example, an isolator or Restricted Access Barrier System (RABS) may be used, and/or processing of the solution from bioburden reducing and/or sterile filtration onwards is performed in a clean room environment, particularly class A clean room environment.

According to the methods described above, the concentration of gellan gum after the respective step (iii), i.e. after the gellan gum is completely dissolved and its concentration is optionally adjusted by further addition of preparatory solvent, is up to about 3 mg/ml. The inventors have found that concentrations higher than about 3 mg/ml gellan gum can create problems with filtration, particularly sterile filtration. For instance, the concentration of gellan gum after step (iii) may be from about 1 mg/ml to about 3 mg/ml. In some embodiments, the concentration of gellan gum after step (iii) is from about 1.0 mg/ml to about 3.0 mg/ml, particularly from about 1.5 mg/ml to about 2.5 mg/ml, more particularly from about 1.8 mg/ml to 2.2 mg/ml, such as about 2.0 mg/ml.

Prior to lyophilization, the method can also encompass a step of filling suitable containers, such as vials or freeze-drying trays, with a suitable volume. In certain embodiments, the container is a vial, particularly a glass vial, or a freeze-drying tray, particularly a disposable freeze-drying tray. Where the container is a vial, it may particularly have a volume of from about 1 ml to about 100 ml, particularly 50 ml. Where the container is a freeze-drying tray, it may particularly have a volume of from about 100 ml to about 2000 ml. After filling, the containers will typically be semi-stoppered or stoppered. Where the method includes a step of sterilizing the composition (e.g. by filter-sterilization), the capping is suitably performed under aseptic conditions as defined herein.

After lyophilization, the method can further encompass a step of capping the container containing the lyophilized composition. Where the method includes a step of sterilizing the composition (e.g. by filter-sterilization), the capping is suitably performed under aseptic conditions as defined herein.

It is encompassed by the invention that at the end of each step, one or more test samples may be taken in order to verify successful conclusion of the previous step by a suitable method. For example, pH and/or concentration samples may be taken, either for information purposes or for adjustments before proceeding with the next step. In certain embodiments, no pH adjustments are performed after step (i).

According to some embodiments, the method of preparing a lyophilized composition as described herein comprises of the following steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration; and
(iv) Lyophilizing the obtained solution.

According to some embodiments, the method of preparing a lyophilized composition as described herein comprises of the following steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iiic) Filling the solution obtained in step (iiib) into a suitable container;
(iiid) Semi-stoppering the container;
(iv) Lyophilizing the solution in the container; and
(v) Capping the container after completion of lyophilization.

In further embodiments, the method does not comprise any other steps than those mentioned above, i.e. the method consists of the steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iiic) Filling the solution obtained in step (iiib) into a suitable container;
(iiid) Semi-stoppering the container;
(iv) Lyophilizing the solution in the container; and
(v) Capping the container after completion of lyophilization.

According to some embodiments, the method of preparing a reconstituted composition as described herein comprises of the following steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iv) Lyophilizing the obtained solution; and
(v) Adding a sufficient amount of pharmaceutically available reconstituting solution, thereby providing the reconstituted gellan gum composition.

According to some embodiments, the method of preparing a lyophilized composition as described herein comprises of the following steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iiic) Filling the solution obtained in step (iiib) into a suitable container;
(iiid) Semi-stoppering the container;
(iv) Lyophilizing the solution in the container;
(v) Capping the container after completion of lyophilization;
(vi) Adding a sufficient amount of pharmaceutically available reconstituting solution, thereby providing the reconstituted gellan gum composition.

In further embodiments, the method does not comprise any other steps than those mentioned above, i.e. the method consists of the steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iiic) Filling the solution obtained in step (iiib) into a suitable container;
(iiid) Semi-stoppering the container;
(iv) Lyophilizing the solution in the container;
(v) Capping the container after completion of lyophilization;
(vi) Adding a sufficient amount of pharmaceutically available reconstituting solution, thereby providing the reconstituted gellan gum composition.

In some embodiments of the invention, the gellan gum content in the reconstituted composition may be from about 2% (w/v) to about 5% (w/v), particularly from about 2.0% (w/v) to about 3.0% (w/v), more particularly about 2.5% (w/v), based on the total volume of reconstituted solution. The reconstituted solution can be used, e.g., in the preparation of a bio-ink, as described below. In this context, it is possible to mix the reconstituted composition with other bio-ink components, such as other polymers and/or living cells. In some embodiments, the reconstituted solution may be mixed with alginate so as to reach an alginate content in the total volume of the final solution is from about 1% (w/v) to about 3% (w/v), particularly from about 1.0% (w/v) to about 2.0% (w/v), more particularly about 1.5% (w/v). Exemplary living cells that may, in some embodiments, be mixed with the reconstituted gellan gum solution are chondrocytes, particularly chondrocytes derived from human chondrocytes, more particularly chondrocytes derived from human auricular chondrocytes.

In a further aspect, the present invention relates to a lyophilized composition comprising gellan gum and a calcium ion sequestrant, wherein the weight ratio of gellan gum to calcium ion sequestrant is from about 15:1 to about 30:1. In particular embodiments, the weight ratio of gellan gum to calcium ion sequestrant is from about 20:1 to about 27:1, more particularly from about 21.5:1 to about 25:1. The lyophilized composition may be prepared according to the method described herein. As already mentioned above with respect to the preparation method, the gellan gum can be high-acyl gellan gum, low acyl gellan gum, or a mixture thereof. In preferred embodiments, low acyl gellan gum is used. The calcium ion sequestrant is as defined hereinabove. In particular embodiments, the calcium ion sequestrant is selected from the group consisting of alkali metal citrates, alkali metal phosphates and alkali metal malates. More particularly, the calcium ion sequestrant is selected from the group consisting of sodium phosphate, potassium phosphate, sodium citrate, potassium citrate and mixtures thereof. In certain preferred embodiments, the calcium ion sequestrant is sodium citrate, particularly tri-sodium citrate dihydrate.

According to the invention, it is preferred that the lyophilized composition is sterile.

It has already been mentioned that the residual moisture content may be used to characterize a lyophilized composition. In some embodiments of the invention, the lyophilized composition has a moisture content of about 5% by weight or less, particularly about 4% by weight or less. In some preferred embodiments, the moisture content will be between about 5% by weight and about 2% by weight. The standard method for determination of residual moisture content is Karl-Fischer titration.

In yet a further aspect, the present invention relates to a dosage-unit formulation comprising a lyophilized composition as defined above in a container with a volume sufficient to allow reconstitution of gellan gum with a pharmaceutically acceptable reconstitution solvent. A sufficient container volume in this context means that there is no need to transfer the lyophilized composition to a more spacious container in order to fabricate the reconstituted solution. Rather, a pharmaceutically acceptable and, in some preferred embodiments, sterile reconstituting solvent can be added to the container with the composition to allow reconstitution to a suitable concentration for downstream applications, such as additive manufacturing. The use of a suitably sized container reduces possible contamination and facilitates handling in downstream use. For example, a single container may be used for lyophilization, reconstitution and optional further steps until the gellan gum composition is, e.g., fed into a 3D bioprinter.

The container may be made from a variety of materials in different three-dimensional forms. In some embodiments, the container is a vial, particularly a glass vial, or a freeze-drying tray, particularly a disposable freeze-drying tray made from plastic. Likewise, the volume of the container may be selected according to the needs of the intended downstream application. Exemplary volumes may range from about 1 ml to about 100 ml, particularly about 50 ml, or from about 100 ml to about 2000 ml.

In particular embodiments, the container is a glass vial with a volume of from about 1 ml to about 100 ml, particularly 50 ml. In other particular embodiments, the container is a disposable freeze-drying tray with a volume of from about 100 ml to about 2000 ml.

As an exemplary dosage unit formulation, a container, particularly a glass vial, containing a lyophilized composition comprising from about 20 mg to about 50 mg gellan gum, or, particularly, comprising from about 20 mg to about 50 mg gellan gum and from about 0.9 mg to about 2.5 mg sodium citrate may be mentioned. This exemplary dosage unit formulation may, e.g., be reconstituted with 1 ml of reconstitution solvent, such as sterile water for injection.

It is also encompassed by the invention to provide a kit comprising a first container, e.g. a vial or a freeze-drying tray, containing a lyophilized gellan gum composition as described herein and a second container, e.g. a syringe, containing a pharmaceutically acceptable reconstitution solvent, particularly water for injection.

In yet a further aspect, the present invention is directed to a sterile composition reconstituted from a sterile lyophilized composition as described hereinabove. In particular embodiments of such sterile reconstituted compositions, gellan gum is low acyl gellan gum and the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate.

In yet a further aspect, the present invention relates to medical uses of the lyophilized composition or the reconstituted composition described herein. According to this aspect, the lyophilized composition or the reconstituted composition are for use in medicine, particularly human medicine.

An exemplary medical use to be mentioned here is regenerative medicine. For instance, the lyophilized composition or reconstituted composition according to the invention may be used in tissue engineering, particularly in additive manufacturing, more particularly in a layer-by-layer deposition method such as bio-printing. Biofabrication techniques based on additive manufacturing bear a huge potential, as they may enable to produce living, patient specific tissues and organs for use in regenerative medicine. These structures can be designed based on clinical 3D models of individual patients to produce personalized tissue grafts. External ear or nose reconstruction is one clinical application that could be significantly improved with bioprinted personalized grafts. Such grafts or implants might even be produced with autologous cells. The reconstituted compositions comprising gellan gum and a reconstituting solvent according to the invention may be used as a component of a so-called bio-ink, i.e. a cell-laden biopolymer formulation, and, as a result, as a component of cross-linked biopolymer formulations or scaffolds or tissue grafts. Bio-inks containing gellan gum are described in EP 20 180 620.5, the content of which is incorporated by reference herein.

Accordingly, the lyophilized composition (after reconstitution) or reconstituted composition according to the invention may be used as a component of a bio-ink and may be mixed with living cells, e.g. chondrocytes. The inventors have surprisingly found that, when using suitable amounts of calcium ion sequestrant, particularly aqueous solutions containing, e.g., from about 1 wt % to about 6 wt %, particularly 3.5 wt % to 4.5 wt % calcium ion sequestrant by weight of the total weight of gellan gum and calcium ion sequestrant, such as sodium citrate, the calcium ion sequestrant does not interfere with printability of such bio-inks or with the viability of the cells due to low pH.

An exemplary medical use of the lyophilized composition or reconstituted composition according to the invention may be as a component of tissue grafts in reconstructive surgery, for example following injuries, surgeries to remove tumors, or to treat birth defects (e.g. anotia, microtia). In this context, the compositions may be used in the manufacturing of the scaffolds of tissue grafts, e.g. by 3D bio-printing.

In further embodiments, the lyophilized composition comprising gellan gum and a calcium ion sequestrant such as sodium citrate as described above is, after reconstitution of the lyophilized composition, for use in sterile applications.

In this context, the invention also relates to the use of a lyophilized composition comprising gellan gum and a calcium ion sequestrant such as sodium citrate, after reconstitution, in sterile applications.

Such sterile applications may be in the field of biotechnology, for example to provide three-dimensional scaffolds for non-medical purposes, e.g. in cell culture.

The invention is further illustrated by the following examples and figures.

### LEGENDS TO THE FIGURES

FIG. 1: Chemical formula of gellan gum repeating unit. (a) High acyl gellan gum; (b) Low acyl gellan gum.

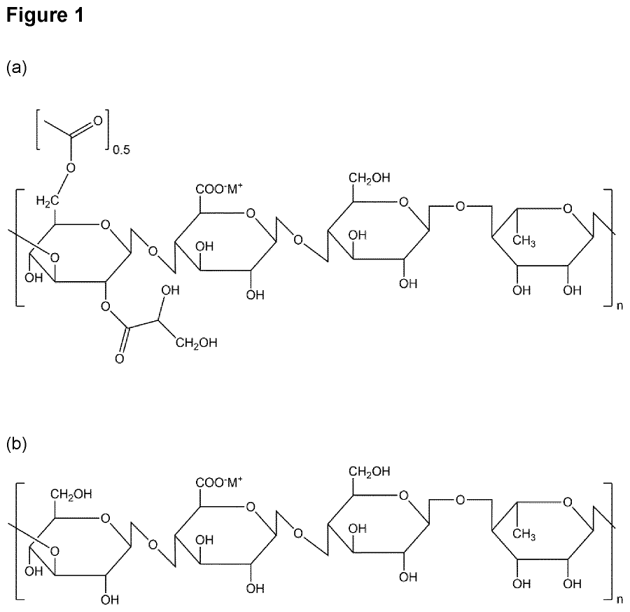

### EXAMPLES

### Example 1: Production of Gellan Gum Without Calcium Ion Sequestrant

Gellan Gum powder (KELCOGEL® CG-LA) is weighed into a powder bag. The Gellan gum is dissolved in water for injection (WFI) so as to reach a final product concentration (at the end of the process) of 2.5 mg/ml. The powder is added gradually to WFI, making sure all the gellan gum will dissolve and no/minimal lumps will occur.

In order to properly dissolve/hydrate the gellan gum, the mixture is heated to ≥80° C. under continuous mixing for half an hour. The solution is stirred and the powder dissolved. After the powder is completely dissolved, the solution is cooled overnight at room temperature under continuous mixing after which a pH and concentration sample is taken for information purposes.

Subsequently, the formulated gellan gum is filtered over a bioburden reducing filter (single filter, 0.2 μm, PES) into a glass bottle into a class A production area (with class B background).

The bioburden reduced gellan gum is then filtered over a sterile filter (single filter, 0.2 μm, PVDF) into a surge tank into a class A production area (with class B background). After filtration, filling into 50 ml glass vials is performed in line with the loading of the freeze dryer. Lyophilization is performed with freezing at atmospheric pressures and temperature steps at −10° C., −50° C., −15° C. and −50° C., primary drying at 100 μbar and temperature steps at −15° C., 0° C. and −10° C., and secondary drying at 100 μbar and temperature steps at +10° C. and +50° C.

After lyophilization, each vial contains 50 mg gellan gum.

### Example 2: Production of Gellan Gum with Calcium Ion Sequestrant

Gellan Gum powder (KELCOGEL® CG-LA) is weighed into a powder bag. The Gellan gum is dissolved in a solution of 0.15 mM sodium citrate trihydrate in water for injection (WFI) per gram gellan gum so as to reach a target concentration (at the end of the process) of 2.0 mg/ml (0.20% w/w). The powder is added gradually to WFI, making sure all the gellan gum will dissolve and no/minimal lumps will occur. After complete addition of the gellan gum, the temperature of the bulk is slightly raised from room temperature to 38° C. to accelerate dissolution. The solution is stirred and the powder dissolved.

After the powder is completely dissolved, a pH and concentration sample is taken for information purposes.

Subsequently, the formulated gellan gum is filtered over a bioburden reducing filter (single filter, 0.2 μm, PES) into a glass bottle. This step is performed in a class C production area.

The bioburden reduced gellan gum is then filtered over a sterile filter (single filter, 0.2 μm, PVDF) into a surge tank into a class A production area. After filtration, filling into 50 ml glass vials is performed in line with the loading of the freeze dryer. Lyophilization is performed with freezing at atmospheric pressures and temperature steps at −10° C., −50° C., −15° C. and −50° C., primary drying at 100 μbar and temperature steps at −15° C., 0° C. and −10° C., and secondary drying at 100 μbar and temperature steps at +10° C. and +50° C.

After lyophilization, each vial contains 40.8 mg gellan gum and 1.8 mg of sodium citrate trihydrate. No issues with undesired gelation or glass vial breakage were encountered.

The invention is further characterized by the following items.
Item 1. A method of preparing a lyophilized gellan gum composition, comprising the steps of:
   (i) Adding gellan gum powder to a pharmaceutically acceptable preparatory solvent;
   (ii) Mixing until substantially complete dissolution;
   (iii) Optionally adding further preparatory solvent;
   (iv) Lyophilizing the obtained solution, thereby providing the lyophilized gellan gum composition;
wherein the concentration of gellan gum after step (iii) is up to about 3 mg/ml.
Item 2. A method of preparing a reconstituted gellan gum composition, comprising the steps of:
   (i) Adding gellan gum powder to a pharmaceutically acceptable preparatory solvent;
   (ii) Mixing until substantially complete dissolution;
   (iii) Optionally adding further preparatory solvent;
   (iv) Lyophilizing the obtained solution;
   (v) Adding a sufficient amount of a pharmaceutically acceptable reconstitution solvent, thereby providing the reconstituted gellan gum composition;
wherein the concentration of gellan gum after step (iii) is up to about 3 mg/ml.
Item 3. The method according to item 1 or item 2, wherein the concentration of gellan gum after step (iii) is from about 1 mg/ml to about 3 mg/ml.
Item 4. The method according to any one of the preceding items, wherein the mixing step (ii) is carried out at a temperature of max. 95° C., particularly at a temperature between about 70° C.-95° C. or at a temperature between about 25° C.-40° C.
Item 5. The method according to any one of the preceding items, further comprising at least one filtration step.
Item 6. The method according to any one of the preceding items, wherein the solution obtained in step (iii) is filtered over a bioburden reducing filter and/or filter-sterilized, before being subjected to lyophilization.
Item 7. The method according to any one of the preceding items, wherein the solution obtained in step (iii) is first subjected to bioburden reducing filtration and then subjected to sterile filtration, thereby providing a sterile solution, before being subjected to lyophilization.
Item 8. The method according to any one of item 6 or item 7, wherein the pore size of the filter used for sterile filtration is 0.22 μm or less, e.g. 0.22 μm or 0.20 μm.
Item 9. The method according to any one of the preceding items, wherein the lyophilization is performed under aseptic conditions.
Item 10. The method according to any one of the preceding items, wherein the gellan gum is low acyl gellan gum, high acyl gellan gum or any mixture thereof, particularly low acyl gellan gum.
Item 11. The method according to any one of the preceding items, wherein the pharmaceutically acceptable preparatory solvent is water or an aqueous solution of a calcium ion sequestrant, particularly an alkali metal citrate, alkali metal phosphate or alkali metal malate solution, more particularly an aqueous sodium citrate solution, more particularly an aqueous solution of tri-sodium citrate dihydrate.
Item 12. The method according to item 11, wherein the concentration of the calcium ion sequestrant in the composition after step (iii) is from about 0.1 mM per g gellan gum powder to about 0.2 mM per g gellan gum powder, particularly about 0.15 mM per g gellan gum powder.
Item 13. The method according to any one of the preceding items, wherein the pharmaceutically acceptable preparatory solvent is an aqueous solution of sodium citrate, and wherein the concentration of sodium citrate in the composition after step (iii) is from about 0.1 mM per g gellan gum powder to about 0.2 mM per g gellan gum powder, particularly about 0.15 mM per g gellan gum powder.
Item 14. The method according to any one of the preceding items, wherein the concentration of gellan gum after step (iii) is from about 1.0 mg/ml to about 3.0 mg/ml, particularly from about 1.5 mg/ml to about 2.5 mg/ml, more particularly about 2.0 mg/ml.
Item 15. The method of preparing a lyophilized composition according to any one of items 1, 3-14, comprising the steps:
   (i) Adding gellan gum powder to a pharmaceutically acceptable preparatory solvent;
   (ii) Mixing until substantially complete dissolution;
   (iii) Optionally adding further preparatory solvent;
   (iiia) Subjecting the solution obtained in step (iii) to bioburden reducing filtration;
   (iiib) Subjecting the solution obtained in step (iiia) to sterile filtration;
   (iv) Lyophilizing the obtained solution;
wherein the concentration of gellan gum after step (iii) is from about 1 mg/ml to about 3 mg/ml.
Item 16. The method of preparing a lyophilized composition according to any one of items 1, 3-14, consisting of the steps:
   (i) Adding gellan gum powder to a pharmaceutically acceptable preparatory solvent;
   (ii) Mixing until substantially complete dissolution;
   (iii) Optionally adding further preparatory solvent;
   (iiia) Subjecting the solution obtained in step (iii) to bioburden reducing filtration;
   (iiib) Subjecting the solution obtained in step (iiia) to sterile filtration;
   (iiic) Filling the solution obtained in step (iiib) into a suitable container;
   (iiid) Semi-stoppering the container;
   (iv) Lyophilizing the solution in the container; and
   (v) Capping the container after completion of lyophilization.
Item 17. The method of preparing a reconstituted gellan gum composition according to any one of items 2-14, consisting of the steps:
   (i) Adding gellan gum powder to a pharmaceutically acceptable preparatory solvent;
   (ii) Mixing until substantially complete dissolution;
   (iii) Optionally adding further preparatory solvent;
   (iiia) Subjecting the solution obtained in step (iii) to bioburden reducing filtration;
   (iiib) Subjecting the solution obtained in step (iiia) to sterile filtration;
   (iiic) Filling the solution obtained in step (iiib) into a suitable container;
   (iiid) Semi-stoppering the container;
   (iv) Lyophilizing the solution in the container;
   (v) Capping the container after completion of lyophilization; and
   (vi) Adding a sufficient amount of a pharmaceutically acceptable reconstitution solvent, thereby providing the reconstituted gellan gum composition.
Item 18. A lyophilized composition comprising gellan gum and a calcium ion sequestrant, wherein the weight ratio of gellan gum to calcium ion sequestrant is from about 15:1 to about 30:1, particularly from about 20:1 to about 27:1, more particularly from about 21.5:1 to about 25:1.
Item 19. The lyophilized composition according to item 18, which is sterile.
Item 20. The lyophilized composition according to item 18 or 19, wherein the gellan gum is low or high acyl gellan gum or any mixture thereof, particularly low acyl gellan gum.
Item 21. The lyophilized composition according to any one of items 18 to 20, wherein the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate.
Item 22. The lyophilized composition according to any one of items 18 to 21, wherein the moisture content of the composition is about 5% by weight or less, particularly about 4% by weight or less.
Item 23. The lyophilized composition according to any one of items 18 to 22, wherein the composition is obtainable by the method of any one of items 1 to 17.
Item 24. A dosage-unit formulation comprising a lyophilized composition according to any one of the preceding items in a container with a volume sufficient to allow reconstitution of gellan gum with a pharmaceutically acceptable reconstitution solvent.
Item 25. The dosage-unit formulation according to item 24, wherein the container is a vial, particularly a glass vial, more particularly a glass vial with a volume of from about 1 ml to about 100 ml, particularly 50 ml.
Item 26. The dosage-unit formulation according to item 24 or item 25, wherein the container is a freeze-drying tray, particularly a disposable freeze-drying tray, more particularly a disposable freeze-drying tray with a volume of from about 100 ml to about 2000 ml.
Item 27. A sterile composition reconstituted from a sterile lyophilized composition, comprising gellan gum and a calcium ion sequestrant, wherein the weight ratio of gellan gum to calcium ion sequestrant is from about 15:1 to about 30:1; wherein the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate; and a pharmaceutically acceptable reconstitution solvent, such as water for injection.
Item 28. The lyophilized composition according to any one of items 18-23 or the dosage-unit formulation according to any one of items 24-26 or the reconstituted composition according to item 27 for use in medicine, particularly human medicine.
Item 29. The lyophilized composition according to any one of items 18-23 for use, after reconstitution of the lyophilized composition comprising gellan gum and a calcium ion sequestrant such as sodium citrate, in sterile applications.
Item 30. Use of a lyophilized composition according to any one of items 18-23 or the dosage-unit formulation according to any one of items 24-26, after reconstitution of the lyophilized composition comprising gellan gum and a calcium ion sequestrant such as sodium citrate, in sterile applications.

1. A method of preparing a lyophilized gellan gum composition, comprising the steps of:
(i) Adding gellan gum powder to a pharmaceutically acceptable preparatory solvent;
(ii) Mixing until substantially complete dissolution;
(iii) Optionally adding further preparatory solvent;
(iv) Lyophilizing the obtained solution, thereby providing the lyophilized gellan gum composition;
wherein the concentration of gellan gum after step (iii) is up to about 3 mg/ml.

2. A method of preparing a reconstituted gellan gum composition, comprising the steps of:
(i) Adding gellan gum powder to a pharmaceutically acceptable preparatory solvent;
(ii) Mixing until substantially complete dissolution;
(iii) Optionally adding further preparatory solvent;
(iv) Lyophilizing the obtained solution;
(v) Adding a sufficient amount of a pharmaceutically acceptable reconstitution solvent, thereby providing the reconstituted gellan gum composition;
wherein the concentration of gellan gum after step (iii) is up to about 3 mg/ml, particularly from about 1 mg/ml to about 3 mg/ml.

3. The method according to claim 1 or claim 2, wherein the mixing step (ii) is carried out at a temperature of max. 95° C., particularly at a temperature between about 70° C.-95° C. or at a temperature between about 25° C.-40° C.

4. The method according to any one of the preceding claims, further comprising at least one filtration step.

5. The method according to any one of the preceding claims, wherein the solution obtained in step (iii) is filtered over a bioburden reducing filter and/or filter-sterilized, before being subjected to lyophilization, particularly first subjected to bioburden reducing filtration and then subjected to sterile filtration, thereby providing a sterile solution, before being subjected to lyophilization.

6. The method according to any one of the preceding claims, wherein the gellan gum is low acyl gellan gum, high acyl gellan gum or any mixture thereof, particularly low acyl gellan gum; and/or
wherein the pharmaceutically acceptable preparatory solvent is water or an aqueous solution of a calcium ion sequestrant, particularly an alkali metal citrate, alkali metal phosphate or alkali metal malate solution, more particularly an aqueous sodium citrate solution, more particularly an aqueous solution of tri-sodium citrate dihydrate;
particularly wherein the concentration of the calcium ion sequestrant in the composition after step (iii) is from about 0.1 mM per g gellan gum powder to about 0.2 mM per g gellan gum powder, particularly about 0.15 mM per g gellan gum powder.

7. The method according to any one of the preceding claims, wherein the pharmaceutically acceptable preparatory solvent is an aqueous solution of sodium citrate, and wherein the concentration of sodium citrate in the composition after step (iii) is from about 0.1 mM per g gellan gum powder to about 0.2 mM per g gellan gum powder, particularly about 0.15 mM per g gellan gum powder; and/or
wherein the concentration of gellan gum after step (iii) is from about 1.0 mg/ml to about 3.0 mg/ml, particularly from about 1.5 mg/ml to about 2.5 mg/ml, more particularly about 2.0 mg/ml.

8. The method of preparing a lyophilized composition according to any one of claims 1, 3-7, comprising the steps:
(i) Adding gellan gum powder to a pharmaceutically acceptable preparatory solvent;
(ii) Mixing until substantially complete dissolution;
(iii) Optionally adding further preparatory solvent;
(iiia) Subjecting the solution obtained in step (iii) to bioburden reducing filtration;
(iiib) Subjecting the solution obtained in step (iiia) to sterile filtration;
(iv) Lyophilizing the obtained solution;
wherein the concentration of gellan gum after step (iii) is from about 1 mg/ml to about 3 mg/ml.

9. A lyophilized composition comprising gellan gum and a calcium ion sequestrant, wherein the weight ratio of gellan gum to calcium ion sequestrant is from about 15:1 to about 30:1, particularly from about 20:1 to about 27:1, more particularly from about 21.5:1 to about 25:1.

10. The lyophilized composition according to claim 9, which is sterile.

11. The lyophilized composition according to claim 9 or 10, wherein the gellan gum is low or high acyl gellan gum; and/or
wherein the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate; and/or
wherein the moisture content of the composition is about 5% by weight or less, particularly about 4% by weight or less.

12. A dosage-unit formulation comprising a lyophilized composition according to any one of the preceding claims in a container with a volume sufficient to allow reconstitution of gellan gum with a pharmaceutically acceptable reconstitution solvent.

13. A sterile composition reconstituted from a sterile lyophilized composition, comprising gellan gum and a calcium ion sequestrant, wherein the weight ratio of gellan gum to calcium ion sequestrant is from about 15:1 to about 30:1; wherein the calcium ion sequestrant is selected from the group consisting of alkali metal citrates, alkali metal phosphates and alkali metal malates, particularly selected from the group consisting of sodium phosphate, potassium phosphate, sodium citrate, potassium citrate and mixtures thereof, more particularly sodium citrate, more particularly tri-sodium citrate dihydrate; and a pharmaceutically acceptable reconstitution solvent.

14. The lyophilized composition according to any one of claims 9-12 or the reconstituted composition according to claim 13 for use in medicine, particularly human medicine.

15. Use of a lyophilized composition according to any one of claims 9-12, after reconstitution of the lyophilized composition comprising gellan gum and a calcium ion sequestrant such as sodium citrate, in sterile applications.
